(19) United States
(12) Patent Application Publication
(10) Pub. No.: US 2023/0157581 A1
(43) Pub. Date: May 25, 2023

(54) METHOD FOR DETERMINING THE METABOLIC PARAMETERS OF A SUBJECT

(71) Applicants: CENTRE NATIONAL DE LA RECHERCHE SCIENTIFIQUE, PARIS (FR); UNIVERSITE DE PARIS, PARIS (FR); MUSEUM NATIONAL D'HISTOIRE NATURELLE, PARIS (FR); SKOLKOVO INSTITUTE OF SCIENCE AND TECHNOLOGY, MOSCOW (RU); ECOLE PRATIQUE DES HAUTES ETUDES, PARIS (FR); SORBONNE UNIVERSITE, PARIS (FR); UNIVERSITE DE PARIS-SACLAY, GIF-SUR-YVETTE (FR)

(72) Inventors: Christophe Goupil, Clichy (FR); Philippe Lecoeur, Bures sur Yvette (FR); Vincent BELS, Pleven (FR); Eric HERBERT, Paris (FR); Henni OUERDANE, Moscow (RU)

(21) Appl. No.: 17/533,479

(22) Filed: Nov. 23, 2021

(51) Int. Cl. A61B 5/11; G16H 20/30; A61B 5/0205

(52) U.S. Cl. CPC A61B 5/1118; G16H 20/30; A61B 5/0205

(57) ABSTRACT

The present invention relates to a method of determining metabolic parameters of one or several muscular motor units by coupling muscular response to ventilatory response of a subject, comprising, from a step of performing efforts (i) using a test device by the subject moving on the test device; (ii)-a step of instantaneously measuring the movement speed of one or several muscular motor units of the subject v(t), and the oxygen flow rate φ_{O2}t); (iii)-a step of plotting the COT curve of the oxygen flow rate of the subject against the movement speed of one or several muscular motor units of the subject v(t), (iv)-a step of determining the following metabolic parameters from the COT curve: Basal power B which is the vertical asymptote at the origin, Flow resistance R_{M} which is the oblique asymptote at high intensities.

### FIELD OF THE INVENTION

The present invention relates to a method of, and a device for characterizing the metabolic response of a subject or an individual to exercise.

State of the Art

The study of the response to exercise is an old subject covering many fields of researches, from physiology, and more specifically sports physiology, to biomechanics.

Energy expenditure has been known and studied since the end of the 19th century, including from a thermodynamic point of view via a calorimetric approach, and then a metabolic and nutritional approach.

Studies on ventilation and ventilatory adaptation are also widespread and are integrated into exercise testing.

The book by the authors Monod, Flandrois, and Vandewalle (Physiologie du sport 6th edition Masson (2007)), makes an exhaustive review of these issues for the humans. Equivalent review can be found, for example, for the horse, in the book “The Athletic Horse, Principles and Practice of Equine Sports Medicine”, edited by David R. Hodgson, Kenneth Harrington McKeever and Catherine M. McGowan.

To date, there is no single protocol for obtaining this characterization directly. The existing measurements and protocols are carried out separately and do not allow a synthetic mapping of the response to exercise of an individual or a subject. It should be noted that the main protocols for identifying an individual's energy potential are based on the following measurements requiring specific equipment:
Ventilation, measured by oxygen Cost of Transport (COT) curves;
Heart rate and rhythm (ECG), and blood gases;
Force-movement speed response of muscles (associated with the Hill 1938 and 1964 model);
Electromyograms.

Moreover, producing a synthetic mapping is only accessible at the cost of multiple protocols on different equipment,

In addition, the publication (Goupil et al. 2019. New J. Phys. 21, 023021 “Thermodynamics of metabolic energy conversion under muscle load”) builds the thermodynamic foundation for the response of a muscle, or a set of muscles, under stress.

It demonstrates that two metabolic parameters can be estimated on the basis of the classical measurement of the force-movement speed response of the muscle: the isometric force Fᵢₛₒ, on the one hand, and a feedback resistance, R_{fb}, on the other hand.

### DISCLOSURE OF THE INVENTION

One purpose of the invention is to allow the quantification of the health condition and performance in terms of energetic potential of an individual from an experimental protocol comprised of two tests. The key innovative character of this invention emerges from the thermodynamic description of the response to exercise, that provide the possibility to bring together the results of these tests in a single descriptive framework.

The method and device of the invention allow for the mapping of a subject's performance and energetic potential in a single test protocol by directly identifying muscular response coupled with ventilatory response of the subject.

To date, there is no single protocol for obtaining this characterization directly. The existing measurements and protocols are carried out separately and do not allow a synthetic mapping of the response to exercise of an individual or a subject.

At the end of the proposed protocol carried out with the device, the synthetic mapping involves 5 traits that are easily calculated:
Basal power, noted B
Threshold metabolic intensity, noted I_{T}
Metabolic resistance, noted R_{M}
Feedback resistance, noted R_{fb}
Isometric force, noted Fᵢₛₒ

All these parameters together give value of metabolic merit factor, Fₘ=(Fᵢₛₒ R_{fb} I_{T})/(B R_{M}), which is the main indicator of the individual's potentiality.

As such, the proposed device can be used both for monitoring sports performance and for functional rehabilitation.

More specifically, the present invention presents a method of determining metabolic parameters of one or several muscle motor units of any subject by coupling muscle response with ventilatory response of the subject.

Comprising the following steps:
(i)+(ii)-a step of acquiring movement speed of one or several muscle motor units of the subject as a function of time v(t), and an oxygen flow rate (φ_{O2}(t) of the subject;
(iii)-a step of plotting the COT curve: the oxygen flow rate against the movement speed of one or several muscle motor units v(t),

the COT curve being coupled to the muscle response by the following equation:

$$COT = \frac{{φ}_{02}}{v} = {a}_{0} + {R}_{m} v + \frac{B}{v}$$

with B the basal power of one or several muscle motor units

R_{M} the flow resistance of one or several muscle motor units
(iv)-a step of determining the following metabolic parameters from the COT curve:
   Basal power B which is the vertical asymptote at the origin;
   Flow resistance R_{M} which is the oblique asymptote;
   a₀ which is the y-intercept of the oblique asymptote.

Indeed, the COT curve has a vertical asymptote at the origin and an oblique asymptote also called the oblique asymptote at high intensities. Alternatively, the step of acquiring a movement speed value of one or several muscular motor units of a subject v(t) and an oxygen flow rate value φ_{O2}(t) can be replaced by a step of performing exercise (i) using a test device and a step of instantaneously measuring (ii) the movement speed of one or several muscular motor units of the subject v(t) and the oxygen flow rate φ_{O2}(t). Alternatively, the step of acquiring may be preceded by a step of instantaneously measuring the movement speed of one or several muscle motor units of the subject v(t), and of the oxygen flow rate φ_{O2}(t) of the subject. In addition, these values can be measured instantaneously during the performance of exercise by the subject performing an exercise test or a stress test on a test device.

### According to Other Optional Features of the Method:

the step (i)+(ii) acquiring also a force F(t),
the step (iii) plotting also a second curve: the force F(t) against movement speed v(t) of one or several muscle motor units,

governed by the following equation from Hill's muscle model:

(F+a) (v+b)=c
in step (iv), the following metabolic parameters are determined from the second curve:
   Isometric force Fᵢₛₒ corresponding to the force at zero movement speed,
   Feedback resistance R_{fb} corresponding to Fᵢₛₒ/vx with vx equal to the movement speed at zero force,
   Threshold metabolic intensity I_{T},
      with the following equations:

$${I}_{T} = b \qquad 1.$$

$${R}_{fb} = \frac{{a}_{0}}{{I}_{T}} = \frac{{a}_{0}}{b} \qquad 2.$$

$${F}_{iso} = \frac{c}{{I}_{T}} - {a}_{0} \qquad 3.$$

Alternatively, the step of instantaneously measuring (ii) the movement speed of one or more muscular motor units of the subject v(t), and of the oxygen flow rate φ_{O2}(t) may be completed by a step of acquiring a value of developed power P(t) and/or force F(t).
in step (iv), the parameters are determined at different times
a figure of merit fₘ is calculated as fₘ=(R_{fb}* I_{T}* Fᵢₛₒ)/(R_{M}*B)
the movement speed of one or several muscle motor units was measured from a test device corresponding to a treadmill or a bicycle
the movement speed of one or several muscle motor units v(t), and the oxygen flow rate φ_{O2}(t) were obtained when the subject was subjected to a variable load Z_{load}(t) which is an instantaneous time-modulated load;
the step (i)+(ii) acquiring also as a function of time a force F(t);
in the step (iv) of determining metabolic parameters of one or several muscle motor units, by:
   calculating Laplace transforms F(p) and V(p) from the force F(t) and movement speed v(t);
   calculating impedances Z(p)=F(p)/V(p);
   calculating, from the impedances Z(p), the following metabolic parameters:
      Isometric force Fᵢₛₒ,
      A resistance impedance of organism of the subject ZIM(p)

The organism being represented by the isometric force and the resistance impedance of the organism such that:

F(p)=Fᵢₛₒ*Z(p)/(Z_{IM}(p)+Z(p))

V(p)=Fᵢₛₒ/(Z_{IM}(p)+Z(p))
Calculating from Z_{IM}(p) the real part which is the metabolic parameter R(I_{M}) and then the following metabolic parameters:
   Feedback resistance R_{fb}
   Threshold metabolic intensity I_{T}
   Basal power B
   Flow resistance R_{M}

Alternatively, the step of acquisition as a function of time may correspond to or be preceded by a step of performing exercise, with the subject moving and being subjected to a variable load which is an instantaneous time-modulated load, as well as to a step of instantaneous measurement, measuring the movement speed of one or several assemblies of muscular motor units, of the developed power or force and of the oxygen flow.
a metabolic power resulting from the difference between input and output powers is modeled,
total mechanical resistance of the output is calculated as

Rᵢₙ=R_{M}+R_{fb}=R_{M}+(Fᵢₛₒ+a₀)/(v_{T}+v),

the measured values being the mechanical force F_{M} and the movement speed v, and

with the force Fᵢₛₒ=F_{B} constant, Rᵢₙ is determined by modulating a load R_{L} (imposed effort)
the test device has real-time controlled braking to produce time-modulated braking over a frequency range of about 10⁻² Hz to 10 Hz, to achieve the load R_{L}
the braking is performed by a motor, mechanically coupled to a driven wheel of the test device, and which acts under active load.
the shape of the time modulation signals of the load R_{L}is:
a time-harmonic, variable-frequency load;
an impulse load;
an indexed load.

According to another embodiment, the invention relates to a device for determining metabolic parameters of one or several muscle motor units coupling muscle response to ventilatory response of a subject, comprising:
means for acquiring efforts coupled to a test device, to measure as a function of time: at least the movement speed of one or several muscular motor units of the subject v(t), and oxygen flow rate φ_{O2}(t);
processor for plotting a COT curve: the oxygen flow rate of the subject against the movement speed of one or several muscle motor units v(t),

the COT curve being coupled to the muscle response by the following equation:

$$COT = \frac{{φ}_{02}}{v} = {a}_{0} + {R}_{m} v + \frac{B}{v}$$

with B the basal power of one or several muscle motor units;

R_{M} the flow resistance of one or several muscle motor units;

the processor being configured to determine the following metabolic parameters from the COT curve:
Basal power B which is the vertical asymptote at the origin;
Flow resistance R_{M} which is the oblique asymptote;
a₀ which is the y-intercept of the oblique asymptote.

According to other optional features of the device:
the means for acquiring are configured to measure a force F(t),
the processor is configured to plot a second curve: the force F(t) against movement speed v(t) of the one or several muscle motor units, governed by the following equation from Hill's muscle model:

(F+a) (v+b)=c
the processor is configured to determine the following metabolic parameters from the second curve:
   Isometric force Fᵢₛₒ corresponding to the force at zero movement speed,
   Feedback resistance R_{fb} corresponding to Fᵢₛₒ/vx with vx equal to the movement speed at zero force,
   Threshold metabolic intensity I_{T},
      With the following equations:

$${I}_{T} = b \qquad 1.$$

$${R}_{fb} = \frac{{a}_{0}}{{I}_{T}} = \frac{{a}_{0}}{b} \qquad 2.$$

$${F}_{iso} = \frac{c}{{I}_{T}} - {a}_{0} \qquad 3.$$

the processor is configured to determine a figure of merit fₘ equal to fₘ=(R_{fb}*I_{T}*Fᵢₛₒ)/(R_{M}*B)
the test device is of the treadmill or bicycle type,
the means for acquiring are configured to measure a force F(t),
the test device is configured to expose the subject to a variable load Z_{load}(t) which is an instantaneous time-modulated load;
the processor is configured to determine metabolic parameters of one or several muscle motor units, by:
   calculating the Laplace transforms F(p) and V(p) from the force f(t) and movement speed v(t);
   calculating the impedances Z(p)=F(p)/V(p);
   calculating, from the impedances Z(p), the following metabolic parameters:
      Isometric force Fᵢₛₒ,
      A resistance impedance of organism of the subject Z_{IM}(p)
         the organism being represented by the isometric force and the resistance impedance of the organism such that:

F(p)=Fᵢₛₒ*Z(p)/(Z_{IM}(p)+Z(p))

V(p)=Fᵢₛₒ/(Z_{IM}(p)+Z(p))
calculating from Z_{IM}(p) the real part which is the metabolic parameter R(I_{M}) and then the following metabolic parameters:
   Feedback resistance R_{fb}
   Threshold metabolic intensity I_{T}
   Basal power B
   Flow resistance R_{M}
the test device has real-time controlled braking to produce time-modulated braking over a frequency range of about 10⁻² Hz to 10 Hz, for example to achieve a load R_{L}
the braking is performed by a motor, mechanically coupled to a driven wheel of the test device, and which acts under active load.

### DESCRIPTION OF THE FIGURES

Other objectives, features and advantages become apparent from the following detailed description with reference to the drawings given for illustrative and non-exhaustive purposes:

FIG. 1 is an example of a force-movement speed response (top), and a power-movement speed response (bottom) for three different muscles: slow muscles (dashed line), medium muscles (dotted line), fast muscles (solid line);

FIG. 2 shows a plot of ϕ− and COT as a function of metabolic intensity for different bodies;

FIGS. 3.a and 3.b show the principle of the invention which allows the determination of metabolic parameters from COT measurement (FIG. 3.a) and from Hill's parametrization (FIG. 3.b);

FIG. 4 shows a diagram of a device according to the invention;

FIG. 5 shows the schematization of the balance between power a) and the associated nodal formalism b);

FIG. 6 shows a synthetic mapping of the metabolic parameters of two individuals or subjects.

### DESCRIPTION OF THE INVENTION

The present invention relates to the determination of intrinsic and extrinsic metabolic parameters of one or several muscular motor units consisting of one or several muscles defining an identity card of a subject's physical condition. The determination is based on:
A thermodynamic model defining the parameters unambiguously.
An exercise measurement device according to an appropriate protocol and parameters extraction.

Each muscular motor unit thus consists of a muscle, and the metabolic parameters are generally determined on several motor units considered.

Preferably, the method according to the invention is implemented by at least one computing means 3, such as a processor 3 associated with a memory. With the one or more processors being adapted to, preferably configured to plot a COT curve :the subject's oxygen flow rate against the subject's movement speed v(t), and to determine metabolic parameters from the curve. In addition, the one or more processors are capable of, preferably configured to execute the various steps of a method according to the invention.

Furthermore, by “means for acquiring” 2a & 2b, is meant, within the meaning of the invention, a test device 1 and/or measurement means. The terms “subject” and “individual” in the present invention, refer to humans but also to any animal species, domestic or wild. In the present invention, the terms “subject” and “individual” are used without distinction.

### THEORETICAL SUMMARY

The core of the modeling is based on metabolic power production by a body (or an organism) and is derived from the publication (Christophe Goupil et al. 2019 New J. Phys. 21, 023021 “Thermodynamics of metabolic energy conversion under muscle load”).

A metabolic power can be broken down into a metabolic force and a metabolic intensity, the product of which gives the metabolic power.

It is considered that the metabolic power is transmitted, via the limbs, in the form of mechanical power and movement speed which are the quantities actually measured experimentally.

The thermodynamic model developed is in perfect agreement with Hill's phenomenological modeling, for which it provides the thermodynamic foundations.

Metabolic power production results from the difference in input, Φ+, and output, Φ−, powers of the organism.

Φ+ corresponds to nutrient supply (mainly glucose), while Φ− is a waste stream that includes excess heat and especially the production of lactic acid-like reaction metabolites.

The point is key, because it follows that the flow Φ− can be considered, to a large extent, proportional to the flow of oxygen consumed by the organism.

The theoretical model leads to the following expressions:

$${Φ}_{+} = \frac{{F}_{iso} {I}_{M} / {η}_{c} + B}{{I}_{T} + {I}_{M}} {I}_{T}$$

$${Φ}_{-} = {R}_{M} {I}_{M}^{2} + \frac{{R}_{fb} {I}_{M}^{2} + {F}_{iso} ( \frac{1}{{η}_{c}} - 1 ) {I}_{M} + B}{{I}_{T} + {I}_{M}} {I}_{T}$$

With:
I_{M}: metabolic intensity (considered proportional to the movement speed of movement of the limb, which is measured);
Fᵢₛₒ: isometric force (extracted from the exercise curves);
n_{c}: maximum thermodynamic efficiency;
B: basal power (extracted from the exercise curves);
I_{T}: threshold metabolic intensity (extracted from the exercise curves).

These concepts were introduced in the publication (Christophe Goupil et al. 2019 New J. Phys.21 023021 “Thermodynamics of metabolic energy conversion under muscle load”).

The metabolic power results from the difference between the input and the output powers, and is written:

$$P = {Φ}_{+} - {Φ}_{-} = {F}_{M} {I}_{M} = [ {F}_{iso} - ( {R}_{M} + \frac{{F}_{iso} + {R}_{fb} {I}_{T}}{{I}_{T} + {I}_{M}} ) {I}_{M} ] {I}_{M}$$

The metabolic power involves two additional terms compared to the previous ones, they are R_{M} and R_{fb}, with:
R_{M}: flow resistance (extracted from the exercise curves) also called the viscous resistance;
R_{fb}: feedback resistance (extracted from the exercise curves).

The five-parameter parameterization thus contains both metabolic (I_{T}, B) and mechanical (F_{iso,}R_{M}) parameters. As regards the term Rfb, it has a metabolic origin but it translates mechanically.

All the parameters can be combined into a metabolic merit factor which is, in a way, the summary scalar of the subject's health condition,

$${f}_{M} = \frac{{R}_{fb} {F}_{iso} {I}_{T}}{{R}_{M} B}$$

All the parameters are now established, there remains to specify some relationships and experimental methods.

Hill's Parameterization

As mentioned above, the metabolic model provided the thermodynamic basis for Hill's muscle model.

Hill proposes a phenomenological expression of the force-movement speed response of one or several muscle motor units, according to:

(F+a) (v+b)=c

### With:

F mechanical force (measured);

v: movement speed of contraction (measured).

The theoretical model developed resulted in the following correspondences:

a=R_{fb}I_{T}+R_{M}I_{M}=a₀+R_{M}I_{M}≈a₀=cte

b=I_{T}=cte

c=(Fᵢₛₒ+R_{fb}I_{T}) I_{T}=cte

The typical force-movement speed response curve can be traced back to the values of the three parameters, since the overall shape is defined by two extreme points, Fᵢₛₒ and the movement speed at zero force.

$${v}_{X} \approx \frac{{F}_{iso}}{{R}_{fb}} .$$

The characteristic dip of the curve defines I_{T}.

The following parameters are therefore extracted:
Fᵢₛₒ
R_{fb}
I_{T}

Balance of the Parameters

We have five parameters:
Fᵢₛₒ: isometric force (extracted from the exercise curves);
B: basal power (extracted from the exercise curves);
I_{T}: threshold metabolic intensity (extracted from the exercise curves);
R_{M}: flow resistance (extracted from the exercise curves);
R_{fb}: feedback resistance (extracted from the exercise curves).

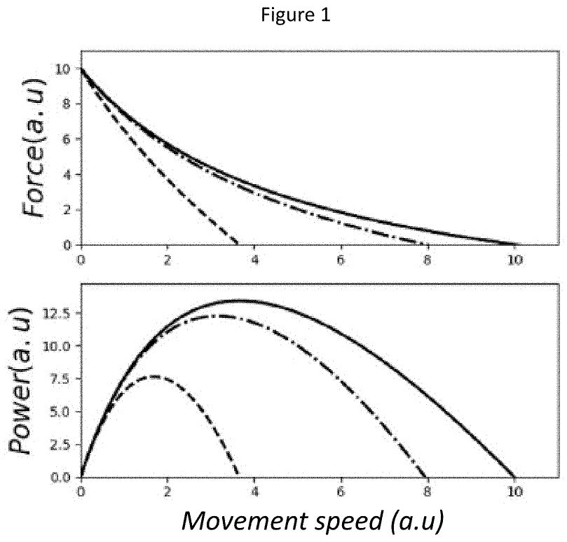

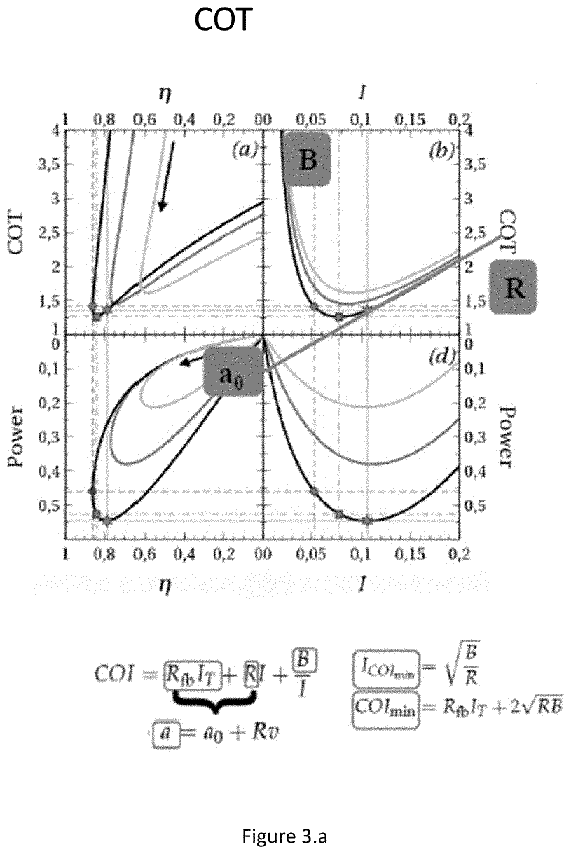

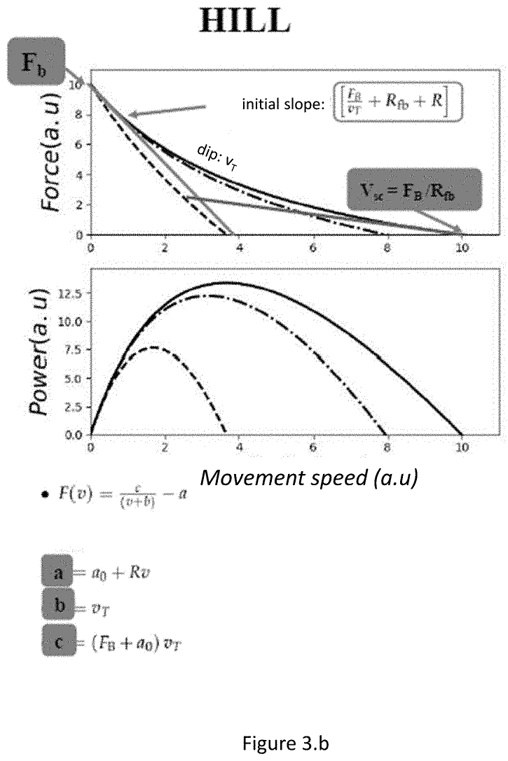

And we have a force-movement speed experiment (FIG. 1 and FIGS. 3.a and 3.b) that determines three of them:

$${I}_{T} = b \qquad 1.$$

$${R}_{fb} = \frac{{a}_{0}}{{I}_{T}} = \frac{{a}_{0}}{b} \qquad 2.$$

$${F}_{iso} = \frac{c}{{I}_{T}} - {a}_{0} \qquad 3.$$

There are still two to be determined. This is the role of the COT measurement.

COI and COT

Using the proportion between the oxygen flow rate and ϕ−, the missing terms can be extracted.

The Cost of Oxygen Index (COI) is defined as:

$$COI = \frac{{Φ}_{-}}{{I}_{M}} = {a}_{0} + {R}_{M} {I}_{M} + \frac{B}{{I}_{M}}$$

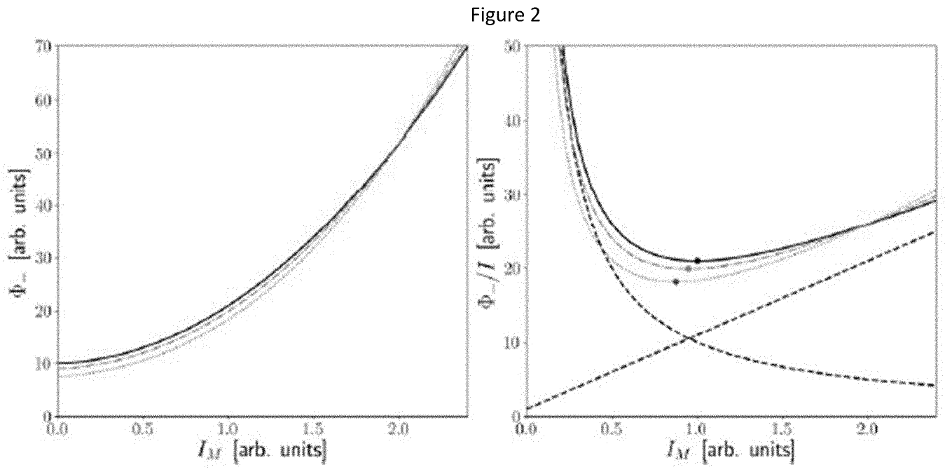

The shape of the plot is shown in FIG. 2.

It should be noted that the curve has a vertical asymptote at the origin, and an oblique asymptote at high intensities. These two asymptotes allow the extraction of, respectively:
B
R_{M}

It should also be noted that that the y-intercept also makes it possible to find Hill's parameter a₀. All five parameters characterizing the organism are therefore accessible.

In the case of movement, and using the proportion between I_{M} and movement speed, the oxygen Cost of Transport (COT) can be defined, which is a quantity widely used in the literature.

In fact, from a dimensional point of view, the COT corresponds to the amount of energy waste produced to move body of the subject over a unit length.

Its expression is:

$$COT = \frac{{φ}_{02}}{v} = {a}_{0} + {R}_{m} v + \frac{B}{v}$$

The form of the COT is therefore isomorphic to that of the COI.

Conclusion: by means of an exercise test measuring powers, movement speeds and oxygen flows, it is possible to calculate the five metabolic parameters that define the mapping of a subject's condition and performance. In addition, the merit factor provides a summary of the subject's state.

### Experimental Embodiment

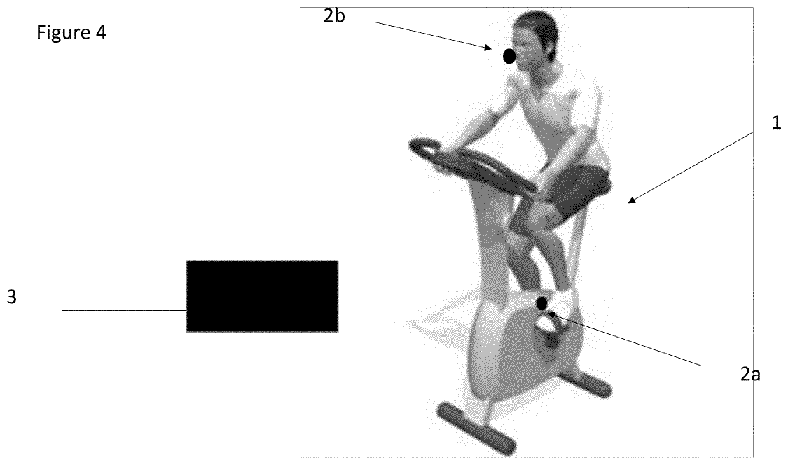

From an experimental point of view, a treadmill or bicycle type exercise test device 1 should be considered, as shown in FIG. 4.

The quantities to be measured will be:
Walking or pedaling movement speed;
Power developed (the force or torque produced will be extracted, knowing the movement speed);
Oxygen consumption.

Protocol

Principle

From a practical point of view, the measurements are equivalent to extracting the mechanical output impedance of the organism.

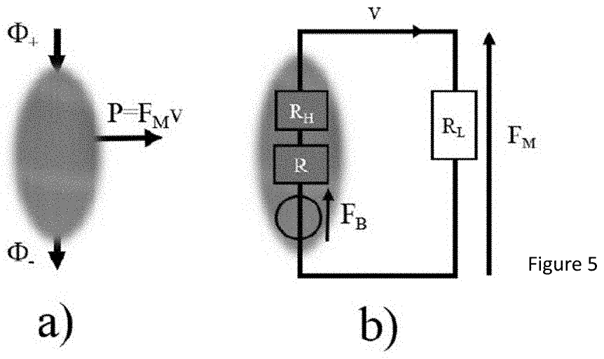

Indeed, the equivalent schematic according to a nodal approach is illustrated in FIG. 5. The one or several limbs, or more precisely the one or several acting muscle units, are assimilated to a source of mechanical power delivering a force, here represented by a generator. In series with the generator are the resistances R_{M} and R_{fb} which induce a power loss by viscous dissipation.

The actual mechanical power available is at the end of the chain. The power results from the product of the force available at the end of the chain, multiplied by the movement speed of movement of the limbs.

The mechanical output resistance is written

Rᵢₙ=R+R_{H}(v)=R_{M}+(Fᵢₛₒ+a₀)/(v_{T}+v)

Knowing that the measured quantities are the mechanical force F_{M} and the movement speed v, on the one hand, and that the force Fᵢₛₒ=F_{B} is constant, on the other hand, the value Rᵢₙ can be extracted by modulating the load R_{L}. Depending on the type of protocol envisaged, the load may be a braking force that prevents movement, or a mass that opposes movement, as in the case of so-called “leg-press” devices.)

### Embodiment

The one skilled in the art will understand that the experimental embodiment can be carried out by means of various devices (treadmill, bicycle, leg press), depending on whether the determination of the metabolic parameters relates to a limb, a portion or all of the organism.

Let us consider the case of a training bike type device, as shown in FIG. 4. The load RL is experimentally achieved by real-time controlled braking to produce time-modulated braking over a frequency range of about 10⁻² Hz-10 Hz. Braking may be performed by a motor, mechanically coupled to the driven wheel, and which acts under active load.

The experimental procedure is equivalent to a harmonic load as encountered in impedance spectroscopy measurements. The electrical analog of the mechanical protocol is the impedance spectroscopy technique used in electrochemistry to determine the performance of electric batteries.

The shape of the modulation signals of the load RL can be of different types, following the tradition of impedance spectroscopy analyses:
a time-harmonic, variable-frequency load;
an impulse load, the load being equivalent to abruptly changing the braking and immediately canceling it (not recommended due to risk of injury);
an indexed load, the load being equivalent to abruptly changing the braking and maintaining it (a technique known as “Wingate” in English), but its exploitation is generally not made beyond the analysis of the shape of the curves).

Thus, at the end of the protocol carried out on the equipment, the synthetic mapping is composed of the following elements:
Basal power;
Chemical energy release rate;
Metabolic, static and dynamic viscosity;
Mechanical, static and dynamic viscosity;
Isometric force.

Measured under stationary stress, these quantities have real values.

On the other hand, when measured under transient stress, the viscosity parameters have an imaginary part which accounts for the elastic and inertial terms.

All of these parameters together are used to obtain the value of the metabolic merit factor, which is the main indicator of the individual's potentiality. As such, the proposed equipment can be used both for monitoring sports performance and for functional rehabilitation. It makes it possible to obtain a synthetic mapping of the response of an individual to exercise, which is not possible to obtain using the state of the art where the measurements and the existing protocols are carried out separately.

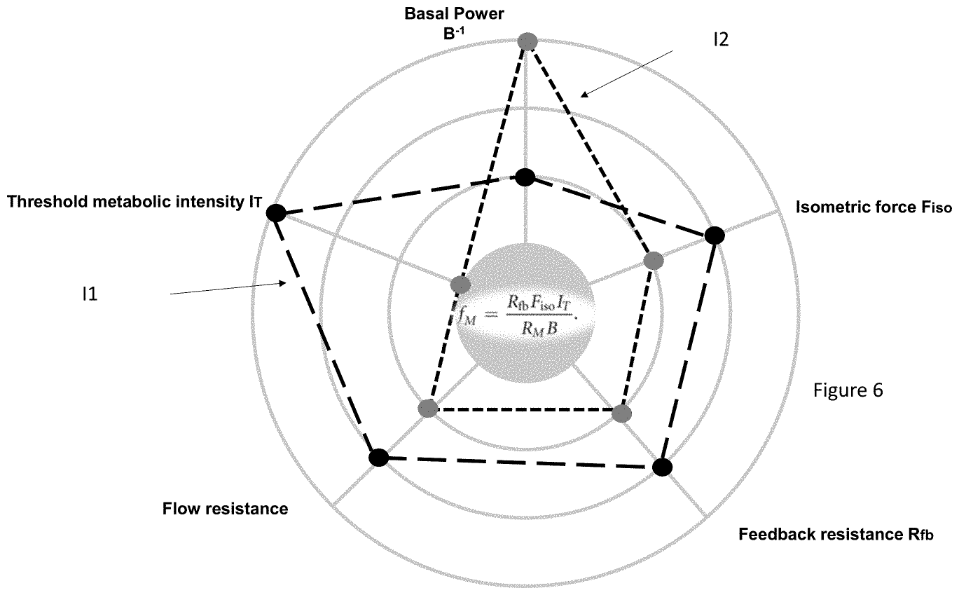

As shown in FIG. 6, two individuals have different values of metabolic parameters.

Individual I1 has a fairly low basal power, high threshold isometric strength and metabolic intensity. These last two quantities show that the individual I1 is capable of subjecting organism of the subject to a significant physical effort. Low basal power of the subject characterizes a dominant of slow type muscles. It is thus an individual adapted to an endurance type effort, rather than an explosive one. The fairly high values of the flow and feedback resistances show that there is room for improvement both physically, through training (lowering of the flow resistance), and possibly metabolically (lowering of the feedback resistance) through better metabolization of nutrients.

Individual I2 has high basal power, low threshold intensity and low isometric strength. The resistances are also of low value. In this case it is about an individual having presumably a very good potential for explosive effort (important Basal), but who is strongly reduced by the weakness of the threshold intensity. This contradiction can be explained by the presence of an injury that does not allow a normally performing body to express its full potential. It can therefore be observed that the collection of the five parameters makes it possible to distinguish between the measured performance and an individual's potentialities.

1. A method of determining metabolic parameters of one or several muscle motor units of a subject by coupling muscle response to ventilatory response of said subject, comprising the following steps:
(i)+(ii)-a step of acquiring movement speed of one or several muscle motor units of the subject as a function of time v(t), and an oxygen flow rate φ_{O2}(t) of the subject;
(iii)-a step of plotting a COT curve of the oxygen flow rate against the movement speed of one or several muscle motor units v(t),
the COT curve being coupled to the muscle response by the following equation:
$$COT = \frac{{φ}_{02}}{v} = {a}_{0} + {R}_{m} v + \frac{B}{v}$$
with
B being the basal power of one or several motor muscle units;
R_{M} being the flow resistance of one or several muscle motor units;
(iv)-a step of determining the following metabolic parameters from the COT curve:
Basal power B which is the vertical asymptote at the origin;
Flow resistance R_{M} which is the oblique asymptote;
a₀ which is the y-intercept of the oblique asymptote.

2. The method according to claim 1, wherein:
the step (i)+(ii) comprises acquiring also a developed force F(t),
the step (iii) comprises plotting also a second curve of the force F(t) against the movement speed v(t) of one or several muscle motor units, governed by the following equation from Hill's muscle model:
(F+a) (v+b=c4
in step (iv), the following metabolic parameters are determined from said second curve:
Isometric force Fᵢₛₒ corresponding to the force at zero movement speed,
Feedback resistance R_{fb} corresponding to Fᵢₛₒ/vx with vx equal to the movement speed at zero force,
Threshold metabolic intensity I_{T},
with the following equations:
$${I}_{T} = b \qquad 1.$$
$${R}_{fb} = \frac{{a}_{0}}{{I}_{T}} = \frac{{a}_{0}}{b} \qquad 2.$$
$${F}_{iso} = \frac{c}{{I}_{T}} - {a}_{0} . \qquad 3.$$

3. The method according to claim 1, wherein in step (iv) the parameters are determined at different times.

4. The method according to claim 2, wherein a factor of merit fₘ is calculated as
fₘ=(R_{fb}*I_{T}*Fᵢₛₒ)/(R_{M}*B).

5. The method according to claim 1, wherein the movement speed of one or several muscle motor units was measured from a test device corresponding to a treadmill or a bicycle.

6. The method according to claim 2, wherein:
the movement speed of one or several muscle motor units v(t), and the oxygen flow rate φ_{O2}(t) were obtained when the subject was subjected to a variable load Z_{load}(t) which is an instantaneous time-modulated load;
the step (i)+(ii) comprises acquiring a developed force F(t);
the step (iv) of determining metabolic parameters of one or several muscle motor units, comprises:
calculating Laplace transforms F(p) and V(p) from the force F(t) and movement speed v(t);
calculating impedances Z(p)=F(p)/V(p);
calculating, from the impedances Z(p), the following metabolic parameters:
Isometric force Fᵢₛₒ,
A resistance impedance of organism of the subject Z_{IM}(p);
the organism being represented by the isometric force and the resistance impedance of the organism of the subject such that:
F(p)=Fᵢₛₒ*Z(p)/(Z_{IM}(p)+Z(p))
V(p)=Fᵢₛₒ/(Z_{Im}(p)+Z(p))
calculating from Z_{Im}(p) the real part which is the metabolic parameter R(I_{M}) and then the following metabolic parameters:
Feedback resistance R_{fb,}
Threshold metabolic intensity I_{T},
Basal power B,
Flow resistance R_{M}.

7. The method according to claim 2, wherein:
a metabolic power resulting from the difference between input and output powers is modeled,
total mechanical resistance of the output is calculated as
Rᵢₙ=R_{M}+R_{fb}=R_{M}+(Fᵢₛₒ+a₀)/(v_{T}+v),
the measured values being the mechanical force FM and the movement speed v, and
with the force Fᵢₛₒ=F_{B} constant, Rᵢₙ is determined by modulating a load R_{L}.

8. The method according to claim 7, wherein the movement speed of one or more several muscle motor units was measured from a test device corresponding to a treadmill or a bicycle, said the test device having a real-time controlled braking to produce time-modulated braking over a frequency range of about 10⁻² Hz to 10 Hz, to achieve the load R_{L}.

9. The method according to claim 8, wherein the braking is performed by a motor, mechanically coupled to a driven wheel of the test device, and which acts under active load.

10. The method according to claim 9, wherein the shape of time modulation signals of the load R_{L} is:
a time-harmonic, variable-frequency load;
an impulse load; or
an indexed load.

11. A device for determining metabolic parameters of one or several muscle motor units of a subject by coupling muscle response to ventilatory response of said subject, comprising:
means for acquiring efforts coupled to a test device, to measure as a function of time: at least movement speed of one or several muscular motor units of the subject v(t), and oxygen flow rate φ_{O2}(t);
a processor for plotting a COT curve of the oxygen flow rate of the subject against the movement speed of one or several muscle motor units v(t),
the COT curve being coupled to the muscle response by the following equation:
$$COT = \frac{{φ}_{02}}{v} = {a}_{0} + {R}_{m} v + \frac{B}{v}$$
with B being the basal power of one or several muscle motor units;
R_{M} being the flow resistance of one or several muscle motor units;
the processor being configured to determine the following metabolic parameters from the COT curve:
Basal power B which is the vertical asymptote at the origin;
Flow resistance R_{M} which is the oblique asymptote;
a₀ which is the y-intercept of the oblique asymptote.

12. The device according to claim 11, wherein:
the means for acquiring are configured to measure a developed force F(t),
the processor is configured to plot a second curve: of the developed force F(t) against movement speed v(t) curve of the one or several muscle motor units, governed by the following equation from Hill's muscle model:
(F+a) (v+b)=c
the processor is configured to determine the following metabolic parameters from the second curve:
Isometric force Fᵢₛₒ corresponding to the force at zero movement speed,
Feedback resistance R_{fb} corresponding to Fᵢₛₒ/vx with vx equal to the movement speed at zero force,
Threshold metabolic intensity I_{T},
With the following equations:
$${I}_{T} = b \qquad 1.$$
$${R}_{fb} = \frac{{a}_{0}}{{I}_{T}} = \frac{{a}_{0}}{b} \qquad 2.$$
$${F}_{iso} = \frac{c}{{I}_{T}} - {a}_{0} . \qquad 3.$$

13. The device according to claim 11, wherein the processor is configured to determine a factor of merit fₘ equal to fₘ=(R_{fb*}I_{T*}Fᵢₛₒ)/(R_{M*}B).

14. The device according to claim 11, wherein the test device is of the treadmill or bicycle type.

15. The device according to claim 11, wherein:
the means for acquiring are configured to measure a force F(t),
the test device is configured to expose the subject to a variable load Z_{load}(t) which is an instantaneous time-modulated load;
the processor is configured to determine metabolic parameters of one or several muscle motor units, by:
calculating Laplace transforms F(p) and V(p) from the force F(t) and movement speed v(t);
calculating impedances Z(p)=F(p)/V(p);
calculating, from the impedances Z(p), the following metabolic parameters:
Isometric force Fᵢₛₒ,
A resistance impedance of organism of the subject Z_{IM}(p)
the organism of the subject being represented by the isometric force and the resistance impedance of the organism such that:
F(p)=Fᵢₛₒ*Z(p)/(Z_{IM}(p)+Z(p))
V(p)=Fᵢₛₒ/(Z_{IM}(p)+Z(p))
calculating from Z_{IM}(p) the real part which is the metabolic parameter R(I_{M}) and then the following metabolic parameters:
Feedback resistance R_{fb};
Threshold metabolic intensity I_{T};
Basal power B;
Flow resistance R_{M}.

16. The device according to claim 15, wherein the test device has real-time controlled braking to produce a time-modulated braking over a frequency range of about 10⁻² Hz to 10 Hz.

17. The device according to claim 16, wherein the braking is performed by a motor, mechanically coupled to a driven wheel of the test device, and which acts under active load.
